(19) United States
(12) Patent Application Publication
(10) Pub. No.: US 2008/0215079 A1
(43) Pub. Date: Sep. 4, 2008

(54) Intraoperative Membrane Cutting Tool

(75) Inventors: Michael S. Collins, San Marcos, CA (US); Tim M. Lehman, Carlsbad, CA (US)

Correspondence Address: FITCH EVEN TABIN AND FLANNERY, 120 SOUTH LA SALLE STREET, SUITE 1600, CHICAGO, IL, 60603-3406, US

(73) Assignee: Zimmer Dental, Inc.

(21) Appl. No.: 11/681,678

(22) Filed: Mar. 2, 2007

(51) Int. Cl. A61B 17/32

(52) U.S. Cl. 606/167

(57) ABSTRACT

A tool for forming a predetermined shaped and sized piece of tissue membrane from a sheet of surgically implantable tissue membrane comprising a body having a cutting portion configured to engage and to form a piece from a sheet of tissue membrane and a drive portion configured to apply a force to the cutting portion to form the piece of tissue membrane. The shapes and sizes of the cutting portions include those typically used in medical procedures.

### FIELD OF THE INVENTION

The present invention relates generally to an intraoperative membrane cutting tool and, more particularly, to a membrane cutting tool for cutting a predetermined shaped and sized piece of tissue membrane from a sheet of surgically implantable tissue membrane.

### BACKGROUND OF THE INVENTION

Tissue membranes are often used in periodontal and dental surgical procedures to replace tissue that a patient has lost or to patch up tissue that was removed or cut into during surgery. The replacement tissue membranes currently used vary widely from a patient's own tissue graft obtained from another part of the body, to human dermis or allografts, to collagen membranes. Regardless of the tissue membrane type used, the person performing the procedure has to select the membrane shape and size to match the area where it is to be applied. Pre-shaped membranes are currently available that represent typical surgical shapes and are sized at an average size that could be used in the average person. However, what usually results is that these pre-shaped and pre-sized membranes are either too large, or inadequately shaped to cover the area, or both. Often what results is the dental practitioner must cut the membrane by hand using scissors or a scalpel to a size and a shape that closely fits the area it is to be applied to. This often requires the practitioner to first remove a sterile tissue sample and to handle it by placing it in or near the mouth of the patient to obtain an accurate estimate of the size and shape desired, and to then further handle the tissue sample by cutting it and possibly placing the tissue sample in or near the mouth one or more times to obtain an accurate shape.

A drawback of doing this type of estimating is that the sterility of the sample may be compromised with all of the increased handling of the membrane. Furthermore, having to cut the membrane sample to a specific size and/or shape before use increases the amount of time and effort spent on shaping the membrane and increases the amount of time spent during the surgery if the membrane can only be shaped just before placement in the patient's mouth. Cutting the membrane often is done by using a scalpel, scissors, or other surgical cutting utensils which may not always allow the clinician the dexterity needed to properly shape the membrane. Additionally, if a larger piece is cut down to a smaller shape and/or size, the excess trimmings of the membrane are often discarded and wasted.

### BRIEF DESCRIPTION OF THE DRAWINGS

FIG. 1 is a perspective view of one embodiment of an intraoperative membrane cutting tool;

FIG. 2 is a bottom plan view of a first handle of the membrane cutting tool of FIG. 1;

FIG. 3 is a perspective view of a second embodiment of an intraoperative membrane cutting tool;

FIG. 4 is a bottom plan view of a first handle of the membrane cutting tool of FIG. 3;

FIG. 5 is a top plan view of a second handle of the membrane cutting tool of FIG. 3;

FIG. 6 is a perspective view of a third embodiment of an intraoperative membrane cutting tool; and

FIG. 7 is a bottom plan view of a first handle of the membrane cutting tool of FIG. 6;

FIG. 8 is a bottom plan view of a first handle and a dial thereon showing placement of a recess in a ball and detent engagement system;

FIG. 9 is a cross-sectional view of the dial and a portion of the first handle shown at FIG. 8;

FIG. 10 is an enlarged view of the circled section 10 shown at FIG. 9;

FIG. 11 is a perspective view of a fourth embodiment of an intraoperative membrane cutting tool;

FIG. 12 is a bottom plan view of a first handle of the membrane cutting tool of FIG. 8;

FIG. 13 is a top plan view of a second handle of the membrane cutting tool of FIG. 8;

FIG. 14 is a perspective view of a fifth embodiment of an intraoperative membrane cutting tool;

FIG. 15 is a bottom plan view of the intraoperative cutting tool of FIG. 11;

FIG. 16 is a perspective view of a sixth embodiment of an intraoperative membrane cutting tool;

FIG. 17 is a bottom plan view of the intraoperative cutting tool of FIG. 13; and

FIG. 18 is a plan view of a tear-drop membrane shape cut with an intraoperative membrane cutting tool.

### DETAILED DESCRIPTION OF THE PREFERRED EMBODIMENTS

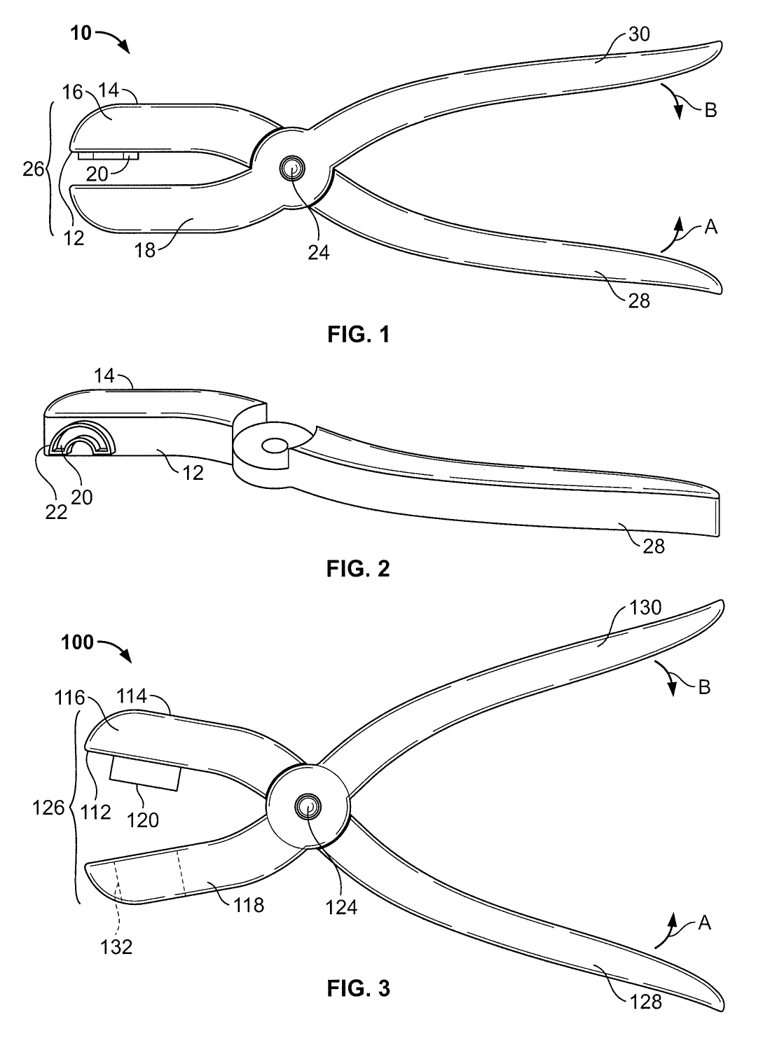

In FIGS. 1 and 2, there is illustrated a cutting tool or membrane cutting device 10. The device 10 includes a jaw 26 defined by a cutting portion 16 and an anvil portion 18. A first handle 28 and a second handle 30 are attached to the jaw portions 16 and 18, respectively, and the handles 28 and 30 are used to open and close the jaw 26. The cutting portion 16 includes a cutting element 20 having a knife-like edge 22 in the shape of a desired membrane shape. To cut a desired membrane shape, a membrane sheet is placed in the jaw 26 between the cutting portion 16 and the anvil portion 18 when opened, and the handles 28 and 30 are then squeezed to close the jaw 26, causing the knife edge 22 to come into contact with the membrane sheet and to cut through the membrane to the anvil portion 18 to form a membrane portion into the desired shape and size.

A set of cutting tools 10 may be provided where each cutting tool 10 has a knife edge 22 configured differently so that the set of tools 10 covers a vast range of shapes and sizes. During a dental procedure, the dental practitioner can instantly select the tool 10 that cuts the desired shape and size membrane portion and make the cut quickly. This eliminates time spent by the practitioner trying to cut the membrane by hand to a needed size and shape with scissors or a knife and provides for a more symmetrical and uniform final product shape than if the membrane were cut by hand. Furthermore, there is less handling of the tissue membrane than with cutting by hand, and therefore, the tissue sterility is better maintained.

As shown in FIG. 1, the cutter portion or cutter body 16 has an inner portion 12 and an outer portion 14 on an opposite side of the cutter body 16. The inner portion 12 includes the cutting element 20, or die, that provides the knife edge 22 about its perimeter, as seen in FIG. 2. The knife edge 22 of the cutting element 20 defines a predetermined shape and size and a hollow recess for receiving the cut portion of the membrane therein. The knife edge 22 of the cutting element 20 projects slightly outward from a bottom surface of the inner portion 12 such that the knife edge 22 is the first part of the inner portion 12 to engage the membrane sheet. Representative shapes and sizes may include any tissue membrane shapes and sizes typically used in dental procedures, and in particular, those shapes include generally C-shaped, I-shaped, tear-drop shaped and crescent shaped. Typical sizes for tissue membranes may be any size that is needed for the procedure, which may be as large as about 30 mm×about 40 mm.

The outer portion 14 of the cutter body 16 is configured to apply force to or drive the inner portion 12 and to move the inner portion 12 into engagement with a membrane sheet for cutting. The outer portion 14 can be manually engaged to press the inner portion 12 of the cutter body 16 into engagement with the sheet of tissue membrane, i.e., by the user applying a direct force by hand to the outer portion 14, or by using the handle 28 to apply a force to the cutter body 16.

The anvil portion 18 is positioned such that it opposes the cutter body 16 and provides a relatively planar surface upon which to support the tissue membrane during cutting operations. The tissue membrane sheet is placed in the jaw 26, i.e., between the cutter body 16 and the anvil portion 18 and is positioned such that it can rest upon the planar surface of the anvil portion 18. When a force is applied to the cutter body 16 to press it into engagement with the membrane sheet, the inner portion 12 of the cutter body 16 is pressed towards the anvil portion 18, such that the cutting element 20 at least partially contacts the planar surface of the anvil portion 18. The knife edge 22 of the cutting element 20 is pressed into and through the membrane sheet until it contacts the flat, planar surface of the anvil portion 18 beneath the sheet, thus cutting the sheet into the predetermined size and shape of the cutting element 20.

Furthermore, the anvil portion 18 is in communication with the second handle 30, where both handles 28 and 30 are connected to each other at a common pivot point 24. The handles 28 and 30 are brought towards one another when each are moved toward one another as indicated by arrows A and B in FIG. 1. As the handles 28 and 30 are moved toward one another, the cutter body 16 and the anvil portion 18 similarly move towards one another. As the cutter body 16 and the anvil portion 18 are brought together, the cutting element 20 is pressed against and through the sheet of tissue membrane and, subsequently, against the planar surface of the anvil portion 18, thus cutting the sheet of tissue into the predetermined shape and size of the cutting element 20 as the knife edge 22 passes through the membrane sheet. The size and length of the handles 28 and 30 can be any size and length as is used in the art. However, the longer the handles 28 and 30 are relative to the corresponding jaw portion 16 and 18 then the more pressure that can be applied to the jaw for cutting to make it easier to cut the sheet of tissue membrane. This can also be advantageous for relatively thicker membrane sheets. The components of the cutting tool are made of sufficiently strong material to perform their functions.

The cutting tool 10 allows the tissue membrane pieces to be easily cut prior to or during the dental procedure. The practitioner first determines the shape and size of the membrane that is needed, preferably a few millimeters larger than the area to be covered, and then selects the cutting tool 10 accordingly to yield the desired shape and size membrane piece.

A membrane sheet then is inserted into the jaw 26 of the cutting tool 10, where the membrane sheet typically is larger than the shape to be cut from it. The handles 28 and 30 are then squeezed together, i.e., by hand, in the direction of the arrows A and B to bring together the cutter body 16 and the anvil portion 18. As the cutter body 16 and the anvil portion 18 are pressed towards each other, the inner portion 12 of the cutter body 16 moves towards the membrane sheet and the knife edge 22 of the cutting element 20 contacts the membrane sheet. The knife edge 22 cuts through the sheet to the anvil portion 18 to form a membrane piece with a similar shape and size as the cutting element 20.

After cutting, the handles 28 and 30 are released and the jaw 26 is able to open to facilitate removal of the tissue sheet and the newly-formed membrane piece. The newly-formed membrane piece is formed into the predetermined shape and size of the cutting element 20. The cut made by the knife edge 22 may be substantially through the sheet of tissue membrane such that the piece is separated from the sheet. The piece of tissue membrane also may become entrapped within the recess of the cutting element 20 after cutting and may need to be removed from the recess, or may be attached very delicately and may need to be pressed out of the lattice of the sheet of tissue membrane to free it from the sheet. The newly-formed membrane piece is then removed from the cutter tool 10 and placed upon the desired area of the patient.

In FIGS. 3-5 there is illustrated a second embodiment of a cutting tool 100. The cutting tool 100 includes a jaw 126 defined by a cutting portion 116 and an anvil portion 118. A first handle 128 and a second handle 130 are attached to each of the jaw portions 116 and 118, respectively, and the handles 128 and 130 are used to open and close the jaw 126. The cutting portion 116 includes a punch 120 having a shape and size of a desired membrane piece. The anvil portion 118 includes a receiving aperture 132 sized and shaped to correspond to that of the punch to receive at least a portion of the punch 120 during cutting operations. To cut a desired membrane shape, a membrane sheet is placed in between the jaw 126 when opened, and the handles 128 and 130 are then squeezed to close the jaw 126, causing the punch 120 to come into contact with the membrane sheet and to cut the membrane in the desired shape and size. More specifically, the punch 120 passes into the receiving aperture 132, where the membrane is sheared at the edge of the receiving aperture 132.

A set of cutting tools 100 may be used, where each cutting tool 100 has a punch 120 configured differently so that a set of tools 100 covers a vast range of shapes and sizes for the membrane piece. During a dental procedure, the dental practitioner can instantly select the tool 100 having the desired shape and size and make the cut quickly. This eliminates time spent by the practitioner trying to cut the membrane by hand, provides for a more symmetrical and uniform final product shape, and requires less handling of the tissue membrane, which promotes better tissue sterility,

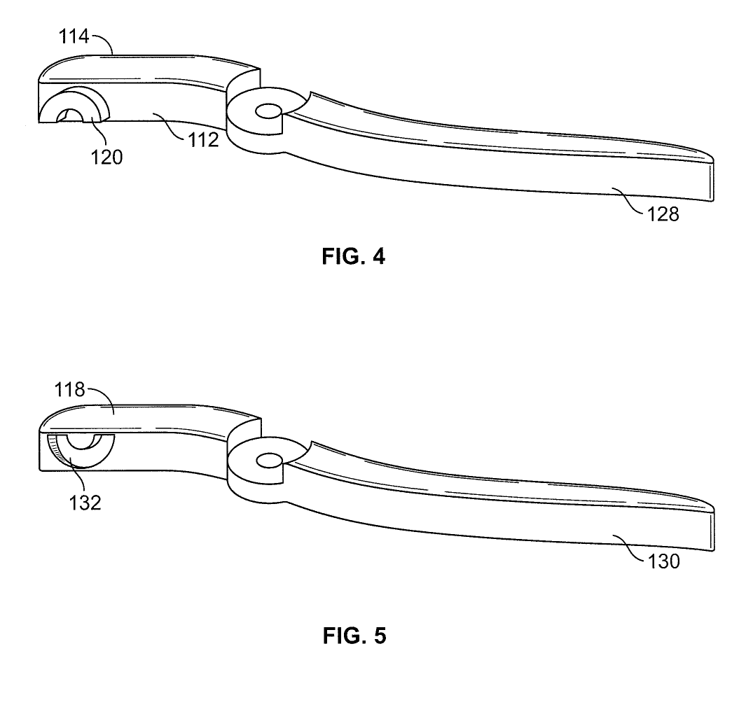

As illustrated in FIG. 3, the cutter body 116 has an inner portion 112 and an outer portion 114 on an opposite side of the cutter body 116. The inner portion 112 includes the punch 120 that extends significantly beyond a bottom surface of the inner portion 112. The punch 120 is preferably not hollow, but rather is a solid body that protrudes outward from the bottom surface of the inner portion 112, as seen in FIG. 4. The punch 120 is configured to have a predetermined shape and size matching that of a desired membrane piece. The punch 120 engages the sheet of tissue membrane and cuts a piece of membrane from the sheet as the sheet passes through the receiving aperture 132. The gap between the punch 120 and the receiving aperture 132, when the punch 120 extends therethrough, is very small in that the sheer force on the membrane at this gap causes the membrane to be sharply cut. The newly-cut membrane piece has the predetermined shape and size similar to that of the punch 120.

The outer portion 114 of the cutter body 116 is configured to apply force to the inner portion 112 and to move the inner portion 112 into engagement with a membrane sheet for cutting. The outer portion 114 can be manually engaged to press the inner portion 112 of the cutter body 116 into engagement with the sheet of tissue membrane, i.e., by the user applying a direct force by hand to the outer portion 114, or by using the handle 128 to apply a force to the cutter body 116.

The anvil portion 118 opposes the cutter body 116 and provides a support surface upon which the tissue membrane can rest on during cutting operations. The anvil portion 118 defines the receiving aperture 132. The anvil portion 118 communicates with the second handle 130 and the cutter body 116 communicates with a first handle 128, where both handles are connected to each other at a common pivot point 124. Preferably, the first handle 128 extends from the cutter body 116, and the second handle 130 extends from the anvil portion 118. The two handles 128 and 130 are brought towards one another as designated by arrows A and B in FIG. 3. As the handles 128 and 130 are moved toward one another, the cutter body 116 and the anvil portion 118 similarly move towards one another. As the cutter body 116 and the anvil portion 118 are brought together, the punch 120 is pressed against the sheet of tissue membrane and subsequently passes into the receiving aperture 132. The shearing force on the membrane at the gap between the punch 120 and the aperture 132, as the punch 120 extends into the aperture 132, causes the membrane to be cut and the desired size and shape of the piece to be pushed through the receiving aperture 132 by the punch 120. The sheet of tissue therefore is cut into the predetermined shape and size of the punch 120. The length of the handles 128 and 130 can be determined by the amount of punching force needed, similar to that described above for the first embodiment.

The cutting tool 100 allows the tissue membrane pieces to be easily cut prior to or during the dental procedure. The practitioner first determines the shape and size of the membrane that is needed, preferably a few millimeters larger than the area to be covered, and then selects the cutting tool 100 with the punch that will yield the desired shape and size membrane piece. Then, a membrane sheet having any standard size is inserted into the jaw 126 of the cutting tool 100. The handles 128 and 130 are then squeezed together, i.e., by hand, in the direction of the arrows A and B to bring together the cutter body 116 and the anvil portion 118. As the cutter body 116 and the anvil portion 118 are pressed towards each other, the inner portion 112 of the cutter body 116 moves towards the membrane sheet. As the inner portion 112 approaches the sheet, the punch 120 contacts the membrane sheet and pushes a portion of the membrane sheet through the receiving aperture 132. The handles 128 and 130 are released and the jaw 126 is allowed to open up to facilitate removal of the tissue sheet and the newly-formed piece.

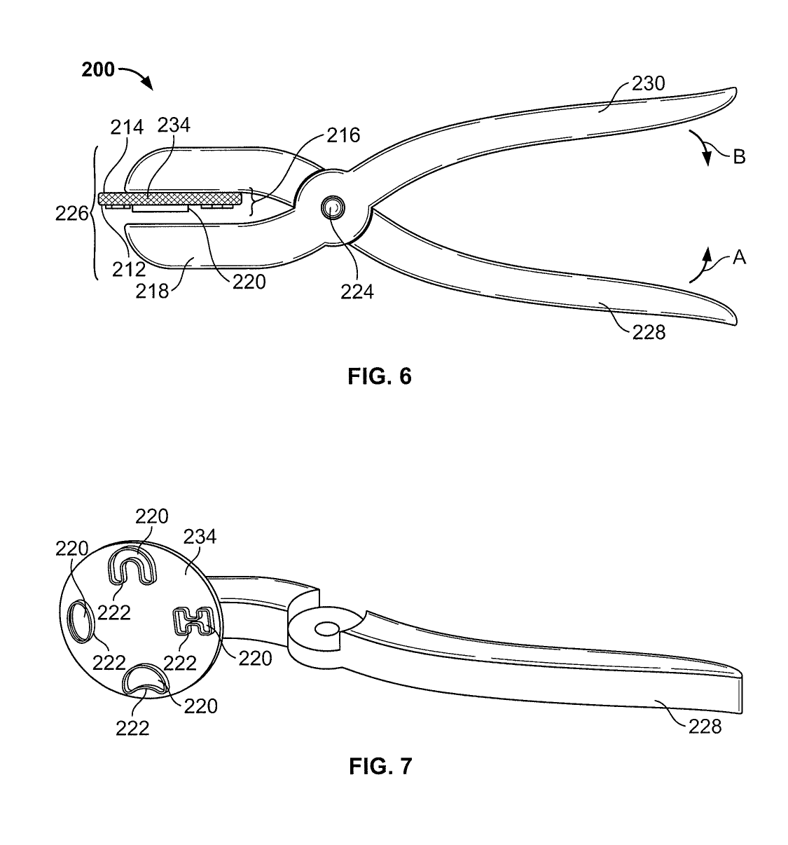

In FIGS. 6 and 7 there is illustrated a third embodiment of a cutting tool 200 having a jaw 226 defined by a plurality of cutters 216 and an anvil 218 generally opposing the cutters 216. A dial 234, or selector plate, includes the cutters 216, each having a knife edge 222 with a perimeter shaped to resemble the size and shape of a different desired tissue membrane piece. A first handle 228 and a second handle 230 operate the jaw portions 216 and 218, respectively, between an open and cutting position. To cut a membrane piece, the size and/or shape is first selected by rotating the dial 234 to shift the desired knife edge 222 into cutting position over the anvil 218. The membrane sheet is placed in the jaw 226 when opened and the handles 228 and 230 are then squeezed to close the jaw 226, causing the knife edge 222 selected on the dial portion 234 to come into contact with the membrane sheet and to cut through the membrane to the anvil 218.

The cutters 216 of the dial 234 each have a different configuration to produce pieces of tissue membrane with different shapes and/or sizes. This arrangement enables a single cutting tool 200 to cover a vast range of shapes and sizes. During a dental procedure, the dental practitioner instantly rotates the dial 234 to select the cutting element 220 having the desired shape and size and then makes the cut quickly. This eliminates time spent by the practitioner trying to cut the membrane by hand to a needed size and shape and provides for a more symmetrical and uniform final product shape than if the membrane were hand cut. Furthermore, there is less handling of the tissue membrane than with hand cutting, and therefore, the tissue sterility is better maintained.

More specifically, the jaw 226 of the cutting tool 200, as shown in FIG. 6, comprises a plurality of cutters 216, or cutter bodies, positioned on the dial 234, and the anvil 218 generally opposing the dial 234. The cutter bodies 216 have a first portion 212 and a second portion 214 on an opposite side of the cutter body 216. The dial 234 includes a plurality of cutter bodies 216, which in turn contain a plurality of cutting elements 220, or dies. Each cutter 216 includes a knife edge 222 having a different shaped and/or sized perimeter from another knife edge 222, as seen in FIG. 7. The dial 234 is rotated at the first handle 228 to position a desired cutter 216 in position to contact and subsequently cut the tissue sheet. The knife edge 222 of each of the cutters 216 is configured to have a predetermined shape and size and further defines a recess for receiving the cut piece of tissue membrane therein. The cutters 216 extend slightly beyond a bottom surface of the dial 214 so to be able to engage a sheet of tissue membrane and to cut through the sheet to the anvil 218 to form a piece of membrane having the desired shape and size. The first handle 228 rotatably attaches to the dial 234 and moves the dial 234 into engagement with a membrane sheet in order to cut it.

The anvil 218 opposes the dial 234 and provides a relatively planar surface upon which to support the tissue membrane during cutting operations. The tissue membrane sheet is placed into the jaw 226, i.e., between the dial 234 and the anvil 218, and is positioned such that it rests on the planar surface of the anvil 218. When a force is applied to the dial 234 to press the selected cutter 216 into engagement with the tissue sheet, the knife edge 222 cuts through the membrane sheet until it contacts the flat, planar surface of the anvil 218 beneath the sheet.

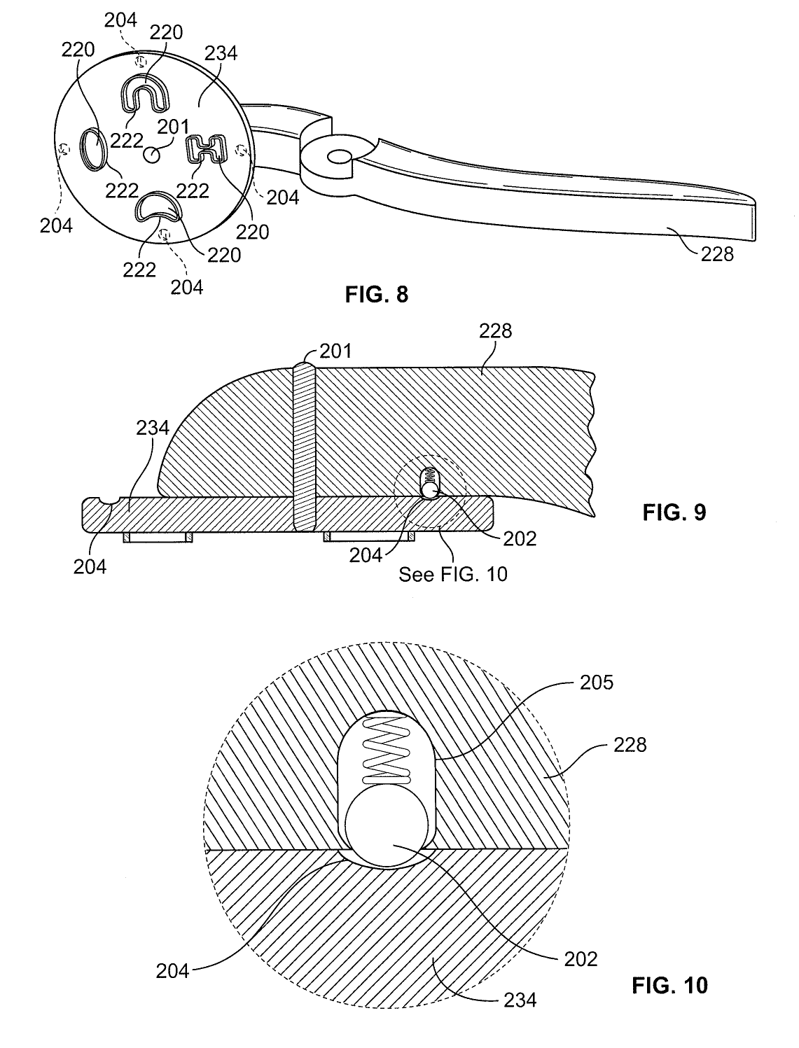

Furthermore, the anvil 218 communicates with the second handle 230 and the cutter body 216 communicates with the first handle 228, where both handles 228 and 230 are connected to each other at a common pivot point 224. Preferably, the first handle extends over the jaw 226 to support the dial 234 in the jaw 226, and the second handle 230 extends from the anvil 218. A central pin 201 attaches the dial 234 to the first handle 228, as shown in FIG. 9. The dial 234 is rotated about the pin 201 to select between the different cutters 216. The dial can have a further engagement with the handle such that it cannot rotate unintentionally. Other locking engagement can be used as well to prevent unintentional rotation of the dial 234. For example, a ball and detent can be used. More specifically, the handle 228 can have a ball 202 that is spring loaded 205 in a socket, as shown in FIGS. 9 and 10. The ball 202 can cam in and out of a recess 204 formed on the dial 234 of each of the cutters, as illustrated in FIGS. 8-10. The size and length of the handles 228 and 230 can be any size and length as is used in the art to address the thickness of the membrane being cut and the overall cutting force required for the cut.

The cutting tool 200 allows the tissue membrane pieces to be easily cut prior to or during the dental procedure. The practitioner first determines the shape and size of the membrane that is needed and then rotates the dial portion 234 of the cutting tool 200 axially, if necessary, to select the cutter 216 that will produce the desired shape and size membrane piece. Then, a membrane sheet is inserted into the jaw 226 of the cutting tool 200. The handles 228 and 230 are then squeezed together, i.e., by hand, in the direction of the arrows A and B to bring together the selected cutter 216 and the anvil 218. As the cutter 216 and the anvil 218 are pressed towards each other, the knife edge 222 of the selected cutter 216 cuts through the membrane sheet to form a membrane piece with a similar shape and size as the selected cutter 216. The handles 228 and 230 are released, and the jaw 226 opens up to facilitate removal of the tissue sheet and the newly-formed piece. The newly-formed membrane piece is removed from the cutter tool 200, similar to the tool 10 in FIG. 1, and placed upon the desired area of the patient.

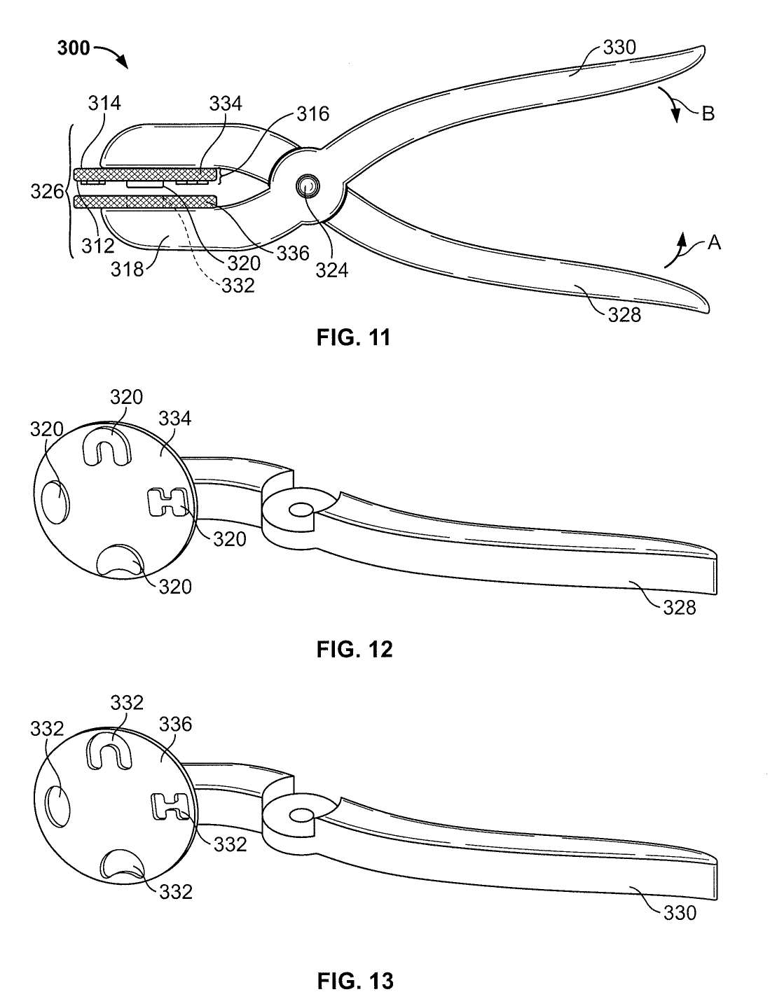

In FIGS. 11-13 there is illustrated a fourth embodiment of a cutting tool 300. The device 300 includes a jaw 326 defined by a punching portion 316 and an anvil portion 318. The punching portion 316 includes a punch dial 334, or selector plate, with a plurality of punch members 320 shaped to resemble the shape of a desired piece of tissue membrane. The anvil portion 318 includes an anvil dial 336 defining a plurality of receiving apertures 332 each configured to receive at least a portion of the selected punch 320 during cutting operations. A first handle 328 and a second handle 330 are attached to each of the jaw portions 316 and 318, respectively. The handles 328 and 330 are used to open and close the jaw 326.

To cut a desired piece of membrane, the desired shape and size of the piece is first selected by rotating the punch dial 334 relative to the first handle 328 to shift a desired punch 320 into position to contact and cut a membrane sheet, while the punch dial 336 is then also rotated relative to the second handle 330 to shift a corresponding receiving aperture 332 into position to receive a portion of the selected punch 320. The membrane sheet is placed in the jaw 326 when opened, and the handles 328 and 330 then are squeezed to close the jaw 326. As the jaw 326 closes, the selected punch 320 contacts the membrane sheet and cuts the membrane into the desired shape and size as the punch extends into the corresponding portion of the membrane sheet which is passed through the aligned receiving aperture 332. The transition between the punch 320 and the corresponding receiving aperture 332 shears the desired piece of tissue from the membrane sheet.

The dials 334 and 336 can have a plurality of punches 320 and corresponding receiving apertures 332, each having a different configuration to produce pieces of tissue membrane with different shapes and/or sizes. This configuration enables a single cutting tool 300 to cover a vast range of shapes and sizes. Representative shapes and sizes may include any tissue membrane shapes and sizes typically used in dental procedures, as discussed above.

During a dental procedure, the dental practitioner can instantly select the desired shape on a single tool 300 and cut the desired piece of tissue quickly. This eliminates time spent trying to manually cut the membrane into a needed size and shape and provides for a more precise final product shape. In addition, this promotes tissue sterility because the tissue is handled less.

The handles 328 and 330 are configured to apply force to its respective dial portion 312 in order to cut the membrane sheet into the desired shape and size. The handles 328 and 330 are used to close the jaw 326. The anvil dial 336 is positioned such that it opposes the punching dial 334 and provides a support surface upon which the tissue membrane can rest on during cutting operations. The support surface of the anvil dial 336 defines the receiving apertures 332 for receiving at least a portion of its corresponding punch 320. More specifically, when a force is applied to close the jaw 326 into engagement with the tissue sheet, the selected punch 320 moves towards its respective receiving aperture 332 and is eventually received at least in part in the receiving aperture 332 during cutting operations. As the selected punch member 320 pushes through the selected receiving aperture 332, it shears the sheet of the tissue membrane as it punches the desired piece through the receiving aperture 332 along with the punch 320 itself. The shearing of the tissue at the edge of the receiving aperture 332 and the outer perimeter edge of the punch 320 cuts the desired piece of tissue. The first and second handles 328 and 330 are connected to each other at a common pivot point 324. The two handles 328 and 330 are pivoted towards one another as designated by arrows A and B in FIG. 11. The handles 328 and 330 are released and the jaw 326 opens up to facilitate removal of the tissue sheet and the newly-formed piece. The newly-formed membrane piece can fall loose from the cutter tool 300 and the tissue sheet or it can be removed from the cutter tool 300 by hand and then placed upon the desired area of the patient.

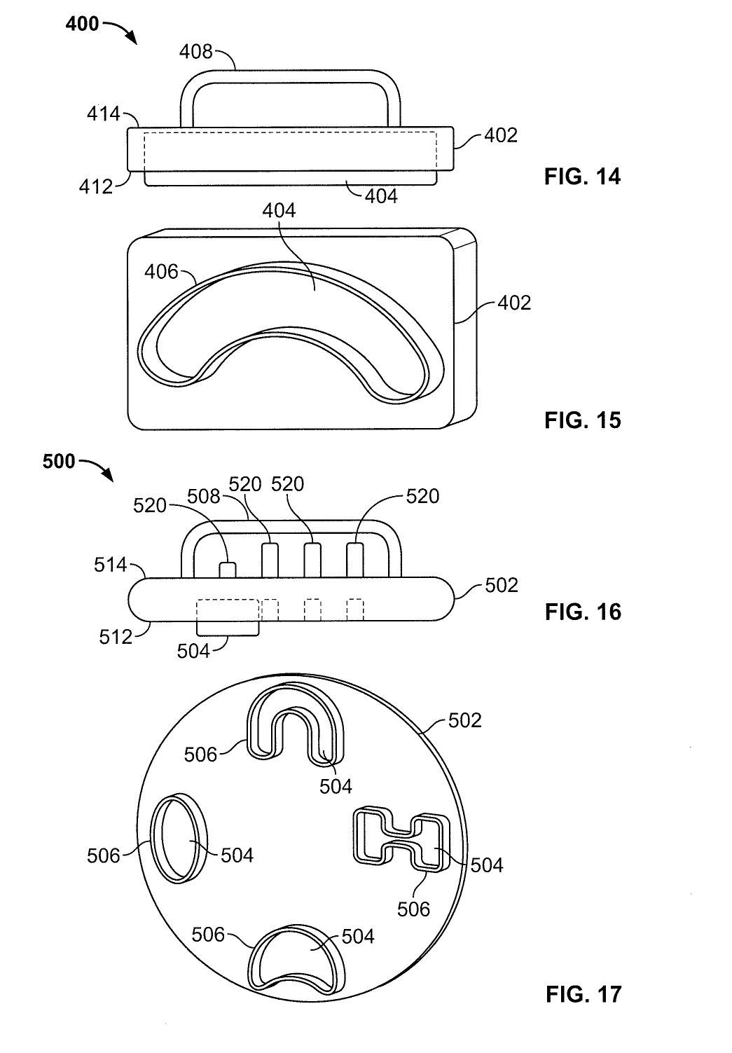

In FIGS. 14 and 15 there is illustrated a fifth embodiment of a cutting tool 400. The cutting tool 400 includes a base 402 with a first portion 412 and a second portion 414 on an opposite side of the base 402. A cutting element 404 extends from the first portion 412 of the base 402. A handle 408 is attached to the second portion 414 of the base 402 and is used to apply a cutting force to the base 402. The cutting element 404 has a knife edge 406 in the shape and size of a desired membrane piece.

The second portion 414 of the base 402 is configured to apply force to the first portion 412 and to move the first portion 412 into engagement with a membrane sheet in order to cut it. The second portion 414 can be manually engaged to press the first portion 412 of the base 402 into engagement with the sheet of tissue membrane, i.e., by the user applying a direct force by hand to the second portion 414, or by using a handle 408 to apply a force to the base 402. The base 402 does not necessarily need to have a handle attached, and if not then the user would apply a force directly to the second portion 414 of the base 402. The cutting element 404 is configured to have a predetermined shape and size and comprises a hollow, interior section for receiving the sheet of tissue membrane therein as it is being cut, the interior section being defined by the upstanding walls of the knife edge 406.

A set of cutting tools 400 may be assembled where each of the cutting tools 400 has a knife edge 406 configured differently so that the set of cutting tools 400 covers a range of shapes and sizes. Representative piece shapes and sizes include any that is typically used in dental procedures. In particular, the shapes generally include, i.e., C-shaped, I-shaped, tear-drop shaped and crescent shaped. Typical sizes for tissue membranes may be as large as up to about 30 mm×40 mm but may be any size that is needed for the procedure. During a dental procedure, the dental practitioner can instantly select the tool 400 having a desired shape and size and cut the membrane into the desired piece quickly. This shortens the cutting time as compared to cutting the membrane by hand and provides for a more precise final product. In addition, because the tissue membrane is handled less, the tissue sterility is improved.

The cutting tool 400 allows the tissue membrane pieces to be easily cut prior to or during the dental procedure. The practitioner first determines the shape and size of the membrane that is needed and then selects the cutting tool 400 accordingly to yield the desired shape and size membrane piece. Then, a membrane sheet is placed beneath the base 402 of the cutting tool 400. A force is applied to a handle 408 such that the base 402 is shifted in a normal direction to the sheet below it. As the base 402 is pressed towards the sheet, the first portion 412 of the base 402 is pressed towards the membrane sheet. As the first portion 412 approaches the sheet, the knife edge 406 of the cutting element 404 contacts the membrane sheet and cuts through the sheet to form a membrane piece with a shape corresponding to the cutting element 404. The cutting tool 400 is then lifted from the sheet by applying a force on the handle 408 in the opposite direction to remove it from the sheet. The piece of tissue is then typically separated from the sheet on its own, or may need to be pressed out of the lattice of the sheet of tissue membrane to free it from the sheet. The piece of tissue membrane may alternatively become entrapped within an interior area of the cutting element 404 and may need to be removed from the interior. The membrane piece is then removed from the cutter tool 400 and placed upon the desired area of the patient. The pieces of tissue membrane can be used for any relevant dental procedure, and in particular, for sinus lifts, tooth extractions, intrabony defects, and periodontal defects. Tissue membrane materials that are commonly used are collagen membranes, allografts, human dermis, and a patient's own tissue via grafting.

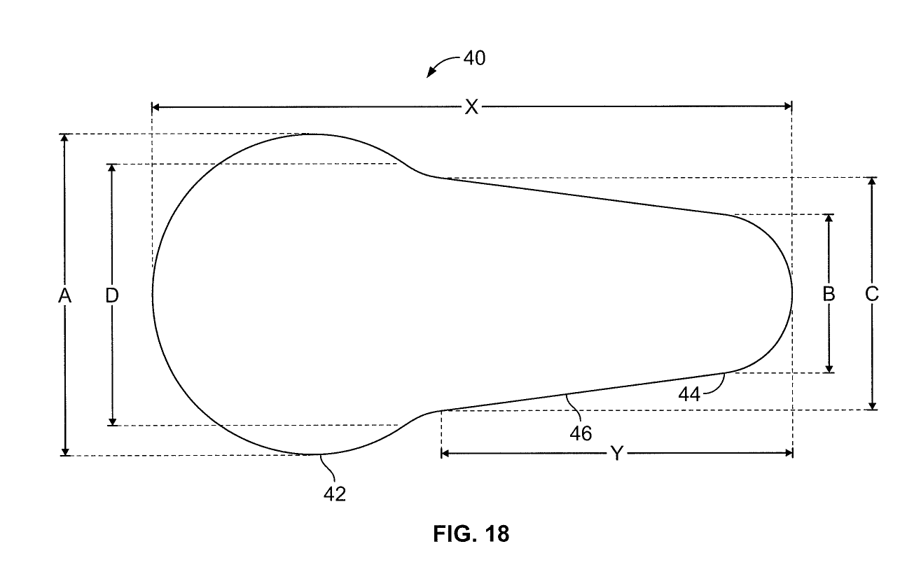

In FIGS. 16 and 17 there is illustrated a sixth embodiment of a cutting tool 500. The cutting tool 500 includes a base 502 defined by a plurality of cutting elements 504, and each cutting element 504 has a first portion 512 and a second portion 514 on an opposite side of the base 502. A plurality of cutting elements 504 extend from the first portion 512 of the base 502. A plurality of selector buttons 520 are positioned at the second portion 514 and are attached to a plurality of cutting elements 504. A single selector button 520 controllably moves its respective cutting element 504 into a cutting position when the selector button 520 is depressed to select the desired cutting element 504. A handle 508 is attached to the second portion 514 of the base 502 and is used to apply a cutting force to the base 502. The plurality of cutting elements 504 each has a knife edge 506 in the shape and size of a desired membrane piece.

The second portion 514 of the base 502 is configured to apply force to the first portion 512 and to move the first portion 512 into engagement with a membrane sheet in order to cut it. The second portion 514 can be manually engaged to press the first portion 512 of the base 502 into engagement with the sheet of tissue membrane, i.e., by the user applying a direct force by hand to the second portion 514, or by using a handle 508 to apply a force to the base 502. The base 502 does not necessarily need to have a handle attached, and if not then the user would apply a force directly to the second portion 514 of the base 502. The cutting elements 504 are configured to have different predetermined shapes and sizes from one another and each cutting element 504 comprises a hollow, interior section for receiving the sheet of tissue membrane therein as it is being cut, which is defined by the upstanding walls of the corresponding knife edge 506.

A single cutting tool 500 may be used which consists of a plurality of cutting elements 504 each having a knife edge 506 with a different configuration than another knife edge 506 to produce pieces of tissue membrane with different shapes and/or sizes so that the single cutting tool 500 covers a range of shapes and sizes. Representative piece shapes and sizes include any that is typically used in dental procedures, and as described in the previous embodiment. During a dental procedure, the dental practitioner can instantly select the cutting element 504 having a desired shape and size on a single tool 500 and cut the membrane into the desired piece quickly. This shortens the cutting time as compared to cutting the membrane by hand and provides for a more precise final product. In addition, because the tissue membrane is handled less, the tissue sterility is improved.

The second portion 514 of the base 502 comprises the selector button or buttons 520 that are connected to at least one of the plurality of cutting elements 504. One button may be configured to control the selection of all of the cutting elements 504, or alternatively, more than one button may be used, and preferably one selector button 520 is used per cutting element 504, as shown in FIG. 16. Where a plurality of selector buttons 520 is used, i.e., one for each cutting element 504, the button attached to the desired cutting element 504 is depressed to select that cutting element 504 and to shift it into an extended position such that the knife edge 506 of the selected cutting element 504 extends into the plane of the tissue membrane. The cutting tool 500 is then shifted in a normal direction to the membrane sheet after force is applied to it such that substantially the entire knife edge 506 of the selected cutting element 504 simultaneously cuts the tissue membrane in the desired shape and size. The other cutting elements 504 which were not selected remain in the non-extended positions, with their respective selector buttons 520 in a non-depressed state.

The cutting tool 500 allows the tissue membrane pieces to be easily cut prior to or during the dental procedure. The practitioner first determines the shape and size of the membrane that is needed and then depresses the selector button 520, if necessary, to select the desired cutting tool 500 to yield the desired shape and size membrane piece. Then, a membrane sheet is placed beneath the base 502 of the cutting tool 500. A force is applied to a handle 508 such that the base 502 is shifted in a normal direction to the sheet below it. As the base 502 is pressed towards the sheet, the first portion 512 of the base 502 is pressed towards the membrane sheet. As the first portion 512 approaches the sheet, the knife edge 506 of the selected cutting element 504 contacts the membrane sheet and cuts through the sheet to form a membrane piece with a shape and size corresponding to the selected cutting element 504. The cutting tool 500 is then lifted away from the sheet by applying a force on the handle 508 in the opposite direction to remove it from the sheet. The piece of tissue is then removed from the cutter tool 500 and placed upon the desired area of the patient. The pieces of tissue membrane can be used for any relevant dental procedure.

In FIG. 18, a representative cut membrane shape is shown. The cut membrane 40 has a generally tear drop shape suitable for use in many surgical procedures. The membrane shape 40 can be cut into the tear drop shape during the surgical procedure using, for example, any of the above preferred tools and methods. The tear drop shaped membrane can be considered to have a first arcuate end 42, with a constant radius of curvature, a second, opposite arcuate end 44 with another, different constant radius of curvature, and an intermediate tapering section 46 extending between the first end 42 and the second end 44. The different radii of curvature render the maximum width A of the first end 42 larger than the maximum width B of the second end 44. For example, the maximum width A of the first end 42 and the maximum width B of the second end 44 can have a predetermined correspondence to one another, such as A=2B. The length X of the membrane and the length Y of the tapering segment also can have a correlation to other dimensions of the membrane, such as X=2A and Y=about 0.5X to about 0.6X.

At the intersection of the tapering segment 46 and the first end 42, there is a relatively small arcuate transition section where the first end 42 is gradually reduced down in width and this reduction can correspond to other dimensions of the membrane. For example, the width D could be about 0.75A to about 0.85A, and the width C could be about 1.40B to about 1.50B. The transition section also can have a constant radius of curvature. For example, such radius of curvature can have a correlation to other dimensions of the membrane, such that the radius of curvature could be in the range of 0.1A to 0.3A.

It will be understood that various changes in the details, materials, and arrangements of parts and components, which have been herein described and illustrated in order to explain the nature of the invention may be made by those skilled in the art within the principle and scope of the invention as expressed in the appended claims.

What is claimed is:

1. A device for cutting a predetermined shaped and sized piece of tissue membrane from a sheet of surgically implantable tissue membrane comprising:
a body having a cutting portion configured to engage a sheet of tissue membrane and cut from the sheet of tissue membrane a piece of tissue with a predetermined shape and size, and a drive portion configured to apply force to the cutting portion to cut the sheet of tissue membrane.

2. The device according to claim 1 wherein the cutting portion is configured with a shape and size that corresponds to a shape and size of a piece of tissue membrane capable of being used in a medical procedure.

3. The device according to claim 1 comprising a plurality of cutting portions, each cutting portion being configured to engage a sheet of tissue membrane and cut from the sheet of tissue membrane a piece of tissue with a different predetermined shape and/or size, and each cutting portion being associated with a drive portion to apply force to the respective cutting portion for cutting the sheet of tissue membrane.

4. The device according to claim 3 wherein each cutting portion is configured with a shape including at least one of a generally C-shaped, I-shaped, tear drop shaped, or crescent shaped.

5. The device according to claim 1 wherein the cutting portion comprises a knife edge and the drive portion is configured to be manually engaged to press the cutting portion into engagement with a sheet of tissue membrane to cut a piece from the sheet of tissue membrane of a predetermined shape and size.

6. The device according to claim 1 further comprising:
at least a first handle communicating with the drive portion of the body and being operable to press the cutting portion of the body into engagement with a sheet of tissue membrane to cut a piece from the sheet of tissue membrane of a predetermined shape and size.

7. The device according to claim 6 further comprising an anvil opposing the body to support the tissue membrane during cutting operations.

8. The device according to claim 7 wherein a second handle communicates with the anvil, and the first and second handles are attached to one another such that movement of the handles towards one another causes the anvil and the body to move towards one another.

9. The device according to claim 8 wherein the first and second handles are attached to one another at a common pivot.

10. The device according to claim 8 wherein the cutting portion of the body can be selected from a plurality of cutting portions and each cutting portion having a different configuration to produce a piece of tissue membrane with a different shape and/or size.

11. The device according to claim 10 wherein each of the cutting portions can be associated with the first handle.

12. The device according to claim 11 wherein each of the cutting portions comprises a knife edge configured to cut from a sheet of tissue membrane a piece of tissue membrane of a different shape and/or size.

13. The device according to claim 10 wherein each of the cutting portions comprises a punch configured to cut from a sheet of tissue membrane a piece of tissue membrane of a different shape and/or size and the anvil defines a space to receive at least a portion of the punch during cutting operations.

14. The device according to claim 13 wherein the space of the anvil is shaped to correspond to the configuration of the punch to cooperate with the punch and produce the desired shape and size of the piece of the tissue membrane removed from the sheet of tissue membrane.

15. The device according to claim 10 wherein the plurality of cutting portions are associated with a selector and the first handle operates to move the selector to position at least one of the cutting portions into engagement with a sheet of tissue membrane to cut a piece of tissue membrane with a predetermined shape and size.

16. The device according to claim 15 wherein the selector is shifted to select one of the plurality of cutting portions.

17. The device according to claim 16 wherein the cutting portions are integral with the selector.

18. The device of claim 16 wherein each of the cutting portions includes a knife edge, and each knife edge being configured to cut from a sheet of tissue membrane a piece of tissue membrane with a different shape and/or size.

19. The device according to claim 16 wherein each of the cutting portions includes a punch extending therefrom and each punch being configured to produce from a sheet of tissue membrane a piece of tissue membrane with a different shape and/or size.

20. The device according to claim 18 wherein the anvil includes a second selector defining a plurality of spaces, each space generally corresponds in shape and size to one of the punches, and receives such corresponding punch at least in part therein during cutting operations.

21. The device according to claim 2 wherein the cutting portion is sufficiently sharp to cut a tissue membrane from the group comprising of collagen membranes, allografts, human dermis, and a patient's tissue via grafting.

22. The device according to claim 21 wherein the cutting portion is configured to cut a piece of tissue membrane in a shape for medical procedures for sinus lifts, tooth extractions, intrabony defects, and periodontal defects.

23. The device according to claim 3 wherein the drive portion is configured to be manually engaged to press at least one of the plurality of cutting portions into engagement with a sheet of tissue membrane and wherein at least one cutting edge of a plurality of cutting edges on the device extends into a plane of tissue membrane while the other cutting edges of the plurality of cutting edges remain in a non-extended position.

24. A method for intraoperatively cutting pieces from a sheet of surgically implantable tissue membrane comprising:
providing a tool with a jaw having a shape for forming from a sheet of a surgically implantable tissue membrane a piece of tissue membrane having a predetermined shape and size;
feeding a sheet of surgically implantable tissue membrane into the jaw of the tool; and
operating the tool to close the jaw to form a piece of tissue membrane with a predetermined shape and size.

25. The method according to claim 24 wherein operating the tool forms a piece of tissue membrane with a shape that is regularly used for medical applications.

26. The method according to claim 25 wherein operating the tool forms a piece of tissue membrane with a shape including at least one of generally C-shaped, I-shaped, tear drop shaped, or crescent shaped.

27. The method according to claim 24 wherein the jaw tool further comprises a support portion configured to engage the shaper and the shaper includes a perimeter shaped corresponding to the predetermined shape of a desired piece of tissue membrane.

28. The method according to claim 27 wherein the tool further comprises a plurality of shapers and further comprises the step of shifting one of the plurality of shapers into alignment with the support portion in the jaw, and each shaper forming a different shape piece of membrane.

29. The method according to claim 28 wherein the support portion further defines a plurality of receiving apertures and each receiving aperture having a shape corresponding to a shape of one of the shapers and further comprises the step of shifting one of the plurality of receiving apertures into alignment with the desired shaper in the jaw.

30. The method according to claim 24 wherein the sheet of surgically implantable tissue membrane is cut with the tool during a surgical procedure for sinus lifts, tooth extractions, intrabony defects, and periodontal defects.

31. The method according to claim 30 wherein the sheet of surgically implantable tissue membrane is cut into a tear drop shape.

32. A method for intraoperatively forming surgically implantable tissue membrane pieces from a sheet of tissue membrane comprising:
placing a cutting edge of a cutting device on top of the tissue membrane sheet, the cutting edge forming at least a generally enclosed shape; and
cutting the tissue membrane by shifting the entire cutting edge in a normal direction relative to a plane generally defined by the tissue membrane so that substantially the entire cutting edge simultaneously cuts the tissue membrane.

33. The method according to claim 32 further comprising:
disposing the cutting device so that at least one cutting edge of a plurality of cutting edges on the cutting device extends into the plane of the tissue membrane while the other cutting edges of the plurality of cutting edges remains in a non-extended position, each cutting edge forming a different shape or size from each other.

34. The method according to claim 32 wherein the tissue membrane sheet is cut with the cutting device during a surgical procedure for sinus lifts, tooth extractions, intrabony defects, and periodontal defects.

35. The method according to claim 34 wherein the tissue membrane sheet is cut into a tear drop shape.
